(19) United States
(12) Patent Application Publication
(10) Pub. No.: US 2016/0176727 A1
(43) Pub. Date: Jun. 23, 2016

(54) APPARATUS FOR UV DISINFECTION OF A LIQUID

(71) Applicant: THE REGENTS OF THE UNIVERSITY OF CALIFORNIA, Oakland, CA (US)

(72) Inventor: Bassam Awni YOUNIS, Davis, CA (US)

(21) Appl. No.: 14/910,652

(22) PCT Filed: Aug. 5, 2014
(86) PCT No.: PCT/US14/49826

§ 371 Date: Feb. 5, 2016

Related U.S. Application Data
(60) Provisional application No. 61/862,460, filed on Aug. 5, 2013.

(51) Int. Cl. C02F 1/32; A61L 2/10

(52) U.S. Cl. CPC C02F 1/325; A61L 2/10; C02F 2201/32

(57) ABSTRACT

An apparatus for disinfecting a liquid using UV radiation comprising a treatment tube in which a liquid vortex with an air core is generated, and a UV light source that is located external to the treatment tube. The air core extends towards the bottom of the treatment tube.

### CROSS-REFERENCE TO RELATED APPLICATIONS

This application claims priority to U.S. provisional application Ser. No. 61/862,460, filed Aug. 5, 2013, which is hereby incorporated by reference in the present disclosure in its entirety.

### BACKGROUND

1. Field

The present disclosure relates to disinfection of liquids, and more specifically to disinfection of liquids using ultraviolet (UV) radiation.

2. Description of Related Art

Water and other liquids need to be disinfected to protect public health. However, current methods have several drawbacks. For example, chlorine disinfection of wastewater is not effective against all pathogens, may produce toxic by-products, and requires care in handling. Conventional UV systems can effectively inactivate pathogens, but may be energy and maintenance intensive, and require high capital costs. Notably, UV radiation can only penetrate a liquid to a certain depth; any liquid that is farther away from the radiation than the penetration depth is not sufficiently irradiated. Some UV systems address this constraint by placing a UV light source within the liquid to be disinfected. However, this approach leads to fouling of the UV light source and higher maintenance costs.

The present disclosure describes an energy-efficient, low-cost UV disinfection apparatus that addresses these constraints.

### BRIEF SUMMARY

The current disclosure describes an apparatus for disinfecting a liquid using UV radiation. In one embodiment, the apparatus includes a treatment tube in which a vortex with an air core is generated. The air core extends towards the bottom of the tube. The liquid is injected tangentially into the tube to form a vortex, irradiated by one or more UV light sources located external to the treatment tube, and collected at the tube outlet.

### DESCRIPTION OF THE FIGURES

FIG. 1 depicts an exemplary treatment tube for disinfection of liquids using UV radiation.

FIG. 2 depicts an exemplary treatment tube for disinfection of liquids using UV radiation.

FIG. 3 depicts an exemplary bleed port used to generate an air core in a treatment tube and various arrangements of UV lamps around the tube.

FIG. 4A depicts a side view of a computer simulated liquid vortex and air core within a treatment tube.

FIG. 4B depicts a top view of a simulated liquid vortex and air core within a treatment tube.

FIG. 5 depicts a computer simulated liquid vortex and air core within a treatment tube showing the UV dose received by the pathogens.

FIG. 6 depicts an exemplary process for disinfecting a liquid using UV radiation.

FIG. 7 depicts an exemplary apparatus for disinfecting a liquid using UV radiation.

FIG. 8 depicts an exemplary apparatus for disinfecting a liquid using UV radiation.

FIG. 9 depicts an exemplary apparatus for disinfecting a liquid using UV radiation.

FIG. 10 depicts experimental results for the breakdown of pharmaceutical compounds in wastewater.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

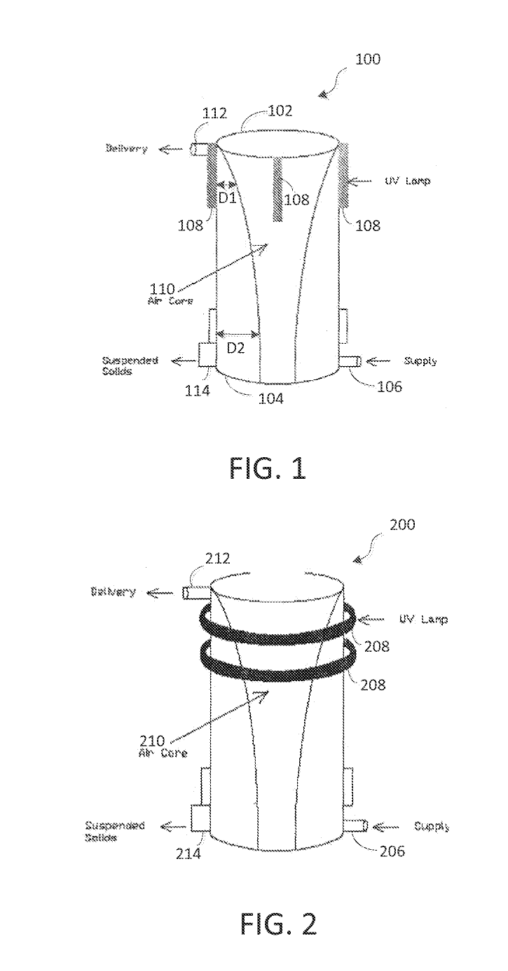

FIG. 1 depicts an exemplary treatment tube 100 for disinfection of a liquid using UV radiation. Treatment tube 100 is a cylinder having a constant radius. In alternative embodiments, the treatment tube may not be cylindrical or may have a non-constant radius.

In some embodiments, the height and radius of the treatment tube may be selected to accommodate a specific flow rate, dwell time, or maximum liquid depth, for example.

In some embodiments, the treatment tube may have a height-to-diameter ratio of 2:1, 3:1, 4:1, 5:1, 6:1, 7:1, or 8:1, for example.

Treatment tube 100 is open at both ends 102, 104. Treatment tube 100 may be placed upon a base during operation to form an enclosure. In alternative embodiments, the treatment tube may have a floor such that it is closed at the bottom of the tube.

The treatment tube may be formed of a material that is transparent or nearly transparent to UV radiation, such as quartz, fused quartz, or synthetic quartz, for example. In some embodiments, the treatment tube may be formed primarily of a material that is not transparent to UV radiation, but includes portions that are transparent to UV radiation. In some embodiments, the treatment tube may be made of robust but not transmissive material (such as aluminum) with slits or openings cut along its length through which UV transmissive strips may be inserted and sealed to prevent leakage. In some embodiments, the treatment tube may include an inner cylinder that is at least partially transparent to UV light and is rotatable, and an outer cylinder that is not transparent to UV light but has one or more cutouts to reveal the inner cylinder. One or more UV light sources may be deposed outside the outer cylinder. In this case, the inner column may be rotated when the exposed area of the inner column becomes fouled or dirty to expose a clean section of the inner column.

In some embodiments, the treatment tube may have reflective materials around it to reflect the UV light back into the tube.

As shown in FIG. 1, treatment tube 100 has a supply inlet 106 in the bottom portion of the tube for injecting a liquid through the side of the treatment tube. In some embodiments, there may be multiple inlets that inject liquid into the treatment tube through the side of the tube at (or near) the bottom of the tube, or through the floor of the tube (if it has a floor), or through nozzles located in any of these positions, or through guide vanes angled so as to introduce the liquid in linear or circular motion. In some embodiments, the supply inlet injects liquid into the tube tangentially such that the liquid is directed onto a circular path around the central axis of the tube. In some embodiments, the supply inlet receives the liquid from a pump. In alternative embodiments, the supply inlet receives the liquid from an elevated reservoir. The flow rate into the inlet may be selected such that a vortex is formed within the treatment tube along the central axis of the treatment tube. The flow rate may depend on the volume of the treatment tube. For example, a treatment tube having a capacity of 5 gallons may receive liquid at a flow rate of 50 gallons/minute.

Treatment tube 100 is depicted with several UV light sources 108 that are located along the treatment tube. The UV light sources are external to the tube and are not in contact with the liquid. In some embodiments, the UV light sources are attached to the treatment tube using a mechanism that holds the UV light sources at a specified distance from the treatment tube. In some embodiments, the mechanism holding the UV light sources may be rotated around the treatment tube to allow repositioning of the UV lights.

The UV light sources may be generated from mercury or Xenon, for example, and may be continuous or pulsed. In some embodiments, each UV light source provides 75 watts of power. In alternative embodiments, each UV light source may provide 25 watts, 50 watts, 100 watts, or 200 watts of power. In some embodiments, the direct (i.e., not reflected) total power density obtainable from the UV light sources may be least 14 W/cm². In other embodiments, the direct total power density may be at least 8 W/cm², 10 W/cm², 12 W/cm², 16 W/cm², or 18 W/cm².

In exemplary treatment tube 100, the UV light sources 108 of treatment tube 100 are straight rods. In alternative embodiments, the UV lights sources may be toroidal light sources that encircle the tube, or the light sources may be helical, or some other geometry. FIG. 2 depicts a treatment tube 200 with toroidal UV light sources 208.

In some embodiments, the UV light sources may be encased in individual channels or in a single enclosure to prevent accidental damage. Some embodiments may include a fan or a number of fans located below the protective channels to cool the UV lamps and purge ozone formed by passage of air over UV lamps.

The UV light sources of treatment tubes 100 and 200 do not extend to the full height of the tube. The UV light sources of treatment tubes 100 and 200 are positioned near the top of the tube, where the depth of the liquid is relatively low due to the larger diameter of the air core 110, 210 (described in more detail below). In some embodiments, the UV light sources may be positioned outside of the tank at locations where the liquid depth is not greater than the penetration depth of the UV radiation. In some embodiments, the UV light sources may extend to the full height of the tube. In some embodiments, there may be only one UV light source.

Treatment tubes 100 and 200 include a delivery outlet 112, 212 that extends outwards from the exterior surface of the tube near the top of the tube, and from which irradiated, disinfected liquid may be collected. Treatment tubes 100 and 200 also include an outlet 114, 214 near the bottom of the tube that may enable removal of solids suspended in the liquid. In alternative embodiments, the treatment tube may not have an outlet for suspended solids. In some embodiments, a treatment tube may include one or more screens or other filters for the removal of the suspended solids separated from the inflow water by the centrifugal forces.

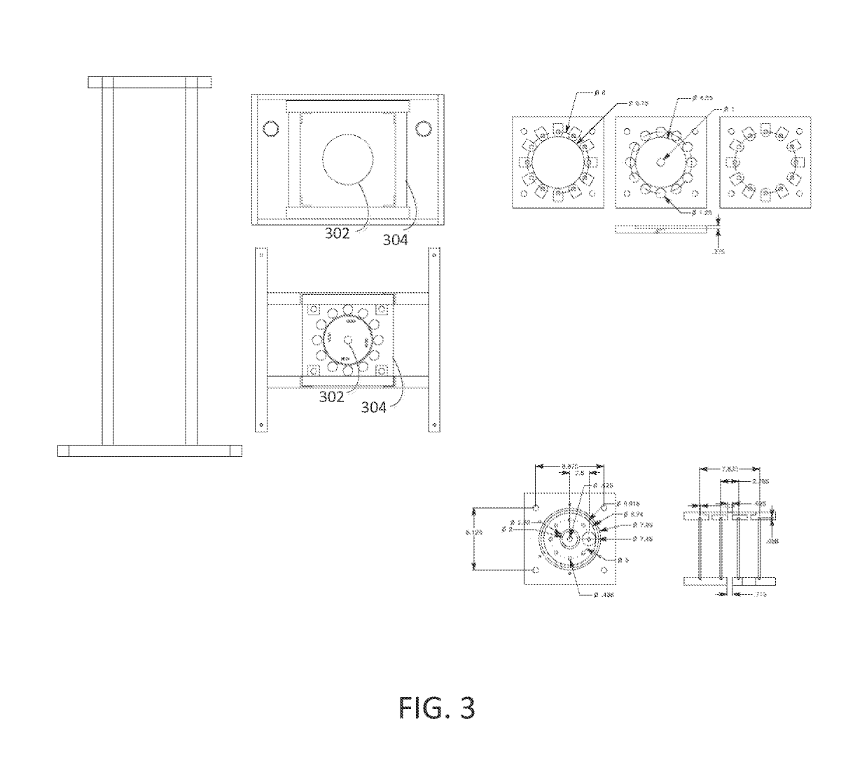

As depicted in FIGS. 1-2, liquid entering the supply inlet forms a vortex with an air core 110, 210 in the center. In some embodiments, the air core may extend to the bottom of the tube. In other embodiments, the air core may extend a quarter of the height of the tube, half the height of the tube, three-quarters of the height of the tube, or may be absent altogether. As will be discussed in more detail with respect to FIG. 3, generation of the air core may be enabled by a bleed port located either in the floor of the treatment tube (if the tube has a floor) or in a base on which the treatment tube is placed during operation (if the tube does not have a floor). The diameter of the bleed port with respect to the geometry of the treatment tube may affect how far down the air core extends towards the bottom of the treatment tube.

The vortex generated in the treatment tube serves to mix the liquid such that all portions of the liquid (and potentially, any suspended solids or slurry) may be exposed to the UV lights located on the sides of the treatment tube. In addition, by adjusting the flow rate and the diameter of the air core, the depth of the liquid (relative to the side of the tube, where the UV light sources are located) may be controlled to ensure that the UV radiation penetrates the liquid. As depicted in FIG. 1, the funnel-shaped air core causes the depth of the liquid D1 at the top of the tube (relative to the sides of the tube) to be less than the depth of the liquid D2 nearer to the bottom of the tube, thereby allowing more effective irradiation at the top. The flow rate may also be adjusted to ensure that the liquid spends a sufficient amount of time in the tube to be effectively disinfected by the UV source lights.

The vortex generated in the treatment tube may also reduce build-up of contaminants, bio-films, or other particles on the interior surface of the treatment tube (fouling), such that it reduces or eliminates the need to suspend operation to clean the tube.

FIG. 3 depicts configurations of a bleed port 302 that may be located in a base 304 on which the treatment tube is placed during operation. The bleed port may allow a small amount of liquid to escape from the tube, thus allowing formation of an air core that extends to the bottom of the tube. In some embodiments, the bleed port may comprise a circular opening that is exposed to the atmosphere. In some embodiments, the diameter of the opening may be selected in relation to the diameter of the tube to enable formation of an air core. In some embodiments, the bleed port may have a raised rim that protrudes above the base. In some embodiments, the liquid escaping through the bleed port is collected and reintroduced into the supply inlet using a Venturi nozzle to create the necessary suction. A valve may be used to control the flow of liquid through the bleed port. The size and extent of the air core in the tube depends on the aperture of this valve. When fully opened, the air core is largest in size and greatest in extent. When fully closed, the air core disappears. Intermediate states are achieved by intermediate apertures.

In some embodiments, if the treatment tube comprises a floor, the bleed port may be located in the floor of the treatment tube rather than in a base on which the treatment tube is placed.

As depicted in FIG. 3, the base may also comprise openings to permit injection of oxidation reagents for ozonation of the liquid, or injection of other gases or chemicals into the liquid. Some embodiments may comprise a mechanism to collect ozone from the top of the protective channels and introduce it into the untreated liquid through perforations in the base.

### 3. Process for Disinfecting a Liquid Using UV Radiation

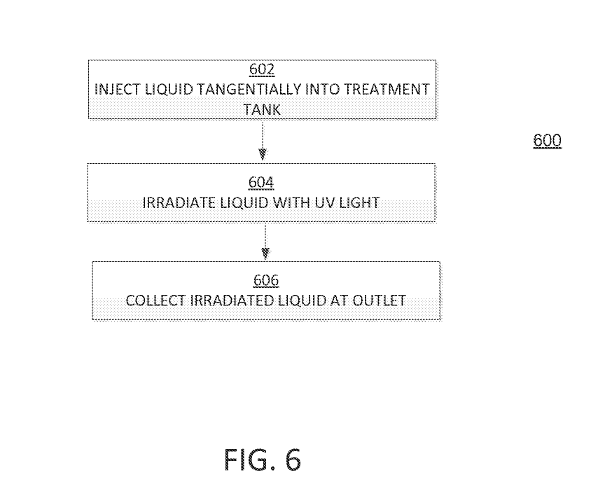

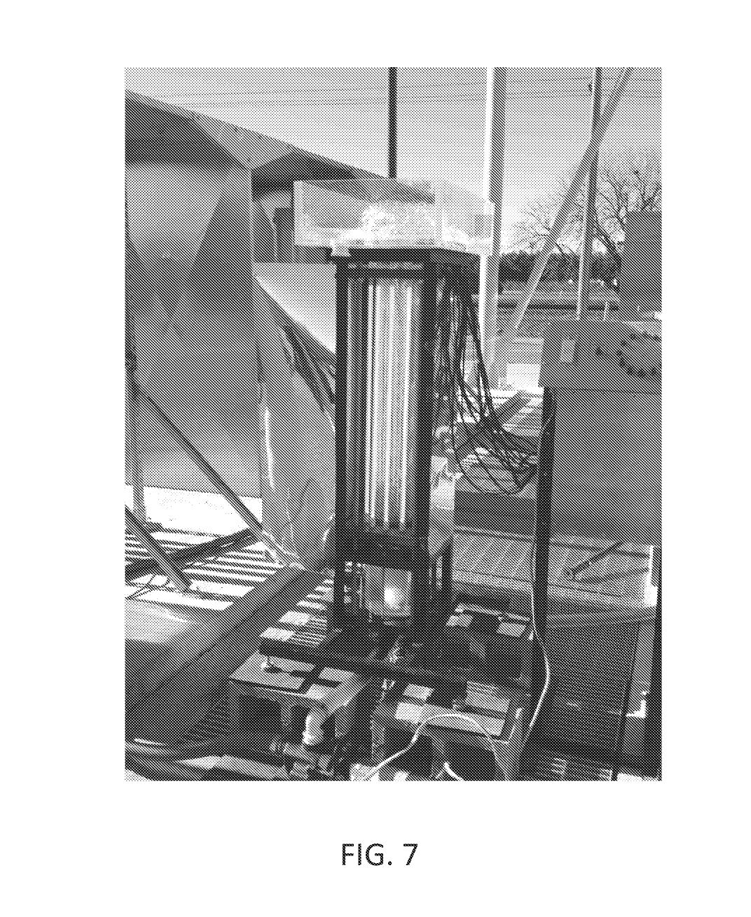

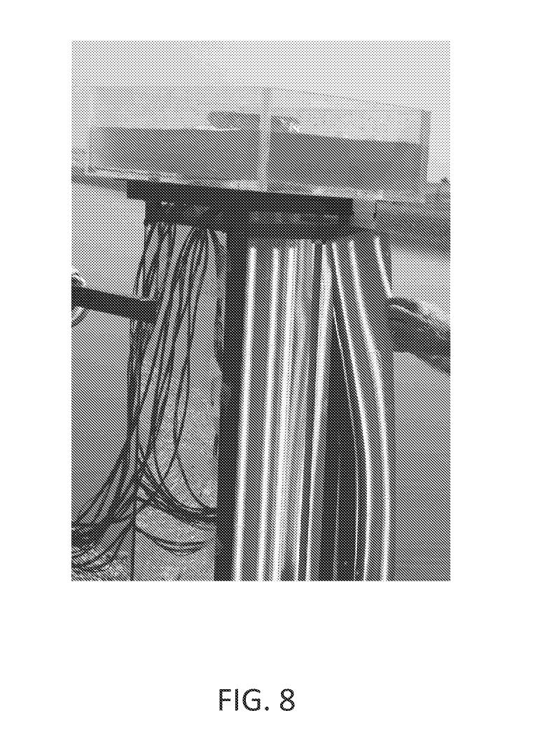

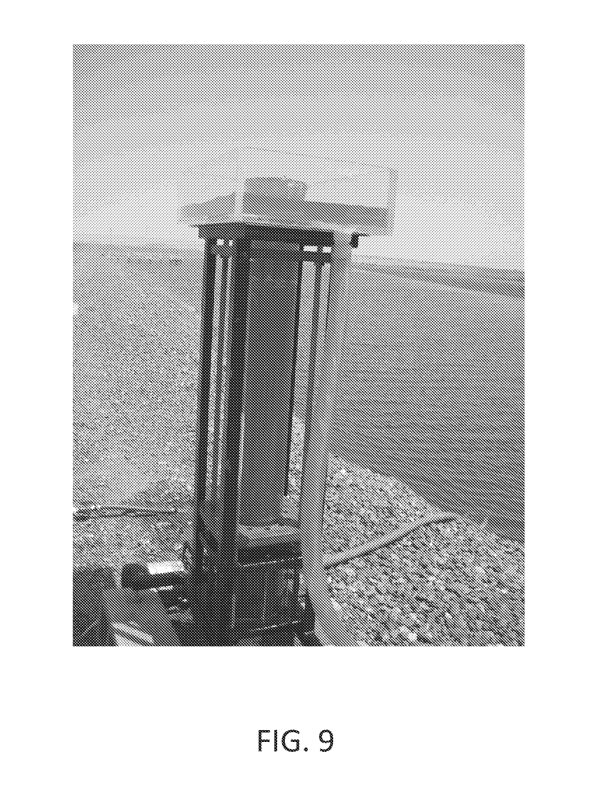

FIG. 6 depicts an exemplary process for disinfecting a liquid using UV radiation.

In block 602, liquid is injected tangentially into a treatment tube. In some embodiments, the liquid is injected through the side of the treatment tube in the bottom portion of the treatment tube. In some embodiments, liquid is injected using apparatus as described earlier with respect to FIGS. 1-2, using a pump and supply inlet. In other embodiments, a pump is not required; for example, if the liquid is at sufficient vertical elevation from the inlet. In some embodiments, the inlet receives the liquid from an elevated reservoir.

In some embodiments, for a small treatment tank having a 5 gallon capacity, liquid may be injected at a rate of 35 gallons per minute, 50 gallons per minute, or 65 gallons per minute. Many other injections rates are possible; the rate of injection is determined in part by the volume of the treatment tube. Larger treatment tanks may have liquid injected at higher rates. In some embodiments, the liquid is injected at a rate such that a vortex is generated in the treatment tube.

In some embodiments, the liquid to be injected contains one or more contaminants. These contaminants may comprise coliforms such as e coli; plant pathogens such as Phytophthora ramorum; pharmaceutical compounds such as NSAID; or insecticides such as pyretheroids, for example.

In block 604, the liquid is irradiated with UV light. In some embodiments, the liquid is irradiated with UV lights configured as described earlier with respect to FIGS. 1-2. In some embodiments, the liquid is irradiated for at least 8 seconds. In other embodiments, the liquid is irradiated for 2 seconds, 4 seconds, 6 seconds, 10 seconds, 12 seconds, 14 seconds, or 16 seconds.

In block 606, the irradiated liquid is collected from an outlet of the treatment tube. In some embodiments, the liquid is collected using apparatus such as described in FIGS. 1-2. In some embodiments, the liquid may be collected in a trough or directed into a pipe. In some embodiments, the irradiated liquid may be suitable for watering crops or for drinking.

### 4. Experimental Results

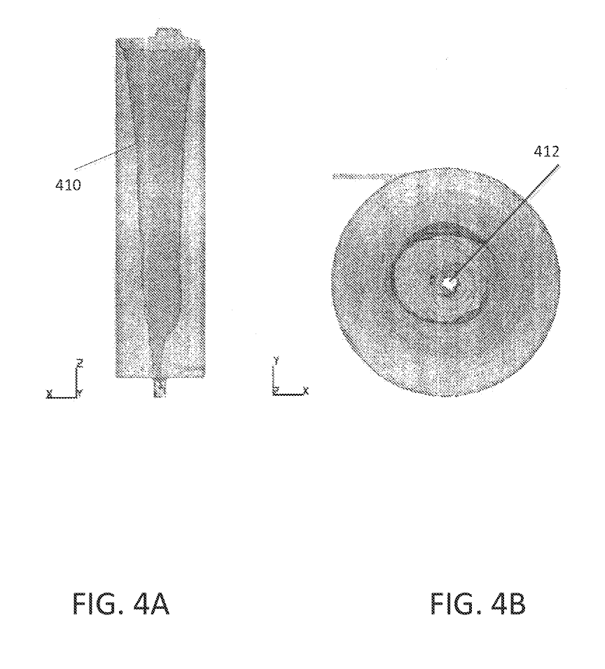

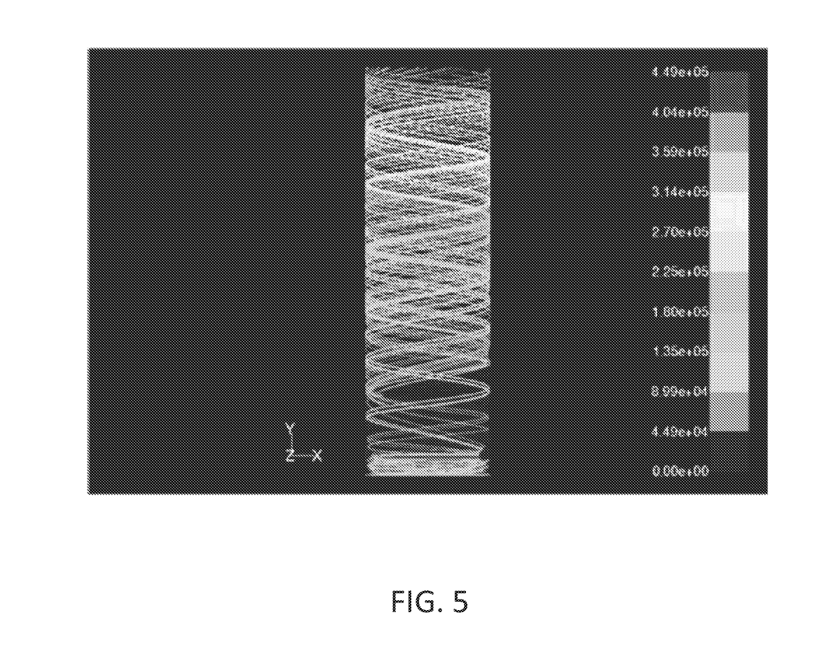

FIGS. 4A-B depict Computational Fluid Dynamic (CFD) simulations of a liquid vortex and air core in a treatment tube. As shown in FIG. 4A, the air core 410 has a funnel shape that is wider at the top of the tube than at the bottom, and the liquid has a correspondingly shallower depth at the top of the treatment tube than at the bottom. The air core in FIG. 4A extends to the bottom of the treatment tube. In alternative examples, the air core may not extend to the bottom of the treatment tube. FIG. 4B depicts a top view of the vortex, showing the centrifugal effect that mixes the liquid. The vortex ‘eye’ 412 is clearly evident in the simulations. FIG. 5 depicts additional simulations of a vortex in a tube showing the UV dose received by pathogens.

With funding from the California Energy Commission, a large-scale vortex reactor was constructed for proof of concept testing. The reactor employs 12 low-pressure mercury UV lamps that are rated at 75 W each. The direct power density obtainable from these lamps is in excess of 14 W/cm² (compared to 3.2 W/cm² obtained in a conventional design). The power density of the vortex reactor is further increased by reflection of UV radiation from four panels of highly-polished aluminum (94% efficiency in reflecting light in the UV-C range) that surround it, thus the total (primary plus reflected) power density is estimated at 18 W/cm². In contrast, none of the UV power reflected off the concrete walls of the conventional reactor is reflected back into the water.

As an initial evaluation of the large-scale reactor, it was installed at the UC Davis Wastewater Treatment Plant. The results for the E. coli bacteria showed disinfection to most probable number (MPN) <2, which is the limit of detection with the US EPA mandated SM 9221 method. Additional test results are shown in Table 1.

A small-scale model of the vortex reactor (having flow capacity of 50 gallons/minute) was constructed and tested over a 14-month period at the UC Davis Waste Water Treatment Plant. The results of these tests were extremely good in that they showed total inactivation of total coliforms (particularly for E. coli) at an energy cost per gallon of water treated that are less than a third of those of the commercial system in operation at UCD.

TABLE 1
Experimental results for total coliform (3 × 5)

| | Most Probable |
|---|---|
| | Number |
| Sample | (MPN)/100 ml |
| Untreated | >1600 |
| water | |
| Sample 1 | 7 |
| (Treated with UV) | |
| Sample 2 | 7 |
| (Treated with UV) | |
| Sample 3 | 4 |
| (Treated with UV) | |

Field tests have shown that disinfection of waste water to the mandated standards for discharge into natural waterways was achieved with treatment tube having height-to-diameter ratio of 4:1 and with a tube diameter to bleed-port diameter ratio of 10. In these tests, waste water was introduced into a treatment tube having a capacity of 5 gallons at a rate of 50 gallons per minute and was irradiated with 4 UV lamps each of power output of 75 W. In computer simulations, disinfection to the mandated standard was found to be achievable with treatment tube height-to-diameter ratios in the range 2:1-8:1 and with tube diameter to bleed-port diameter ratios in the range 8-12.

The typical dwell time of wastewater flowing in the treatment tube at rate of 50 gallons per minute was calculated to be around 10 seconds. Typical UV penetration depth is estimated at 3.5 inches. The delivered dose (calculated as the product of the UV intensity times the exposure time) was calculated as 575 J/m2, producing a log inactivation of 2.69.

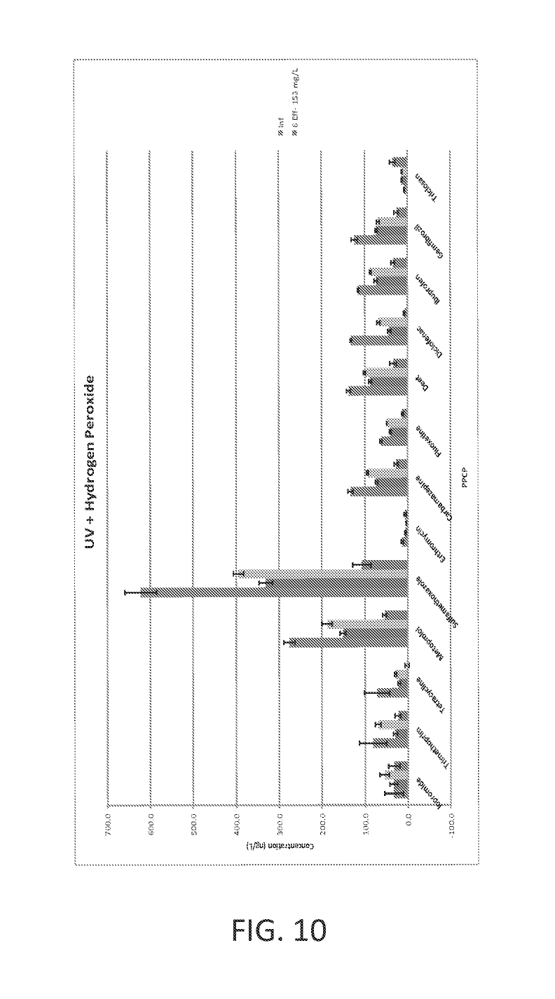

Further tests have been performed in which an oxidizing agent (H₂O₂) was introduced into the untreated water before being exposed to the UV light. Here again the results were extremely good: the combination of UV and H₂O₂ eliminated pharmaceuticals and other contaminants that are normally left untreated by the conventional methods. Test results are depicted in FIG. 10. The pharmaceutical compounds tested are indicated on the horizontal axis. For each compound, four bars are shown. The first bar on the left represents the concentration of that particular compound before irradiation. Each subsequent bar represents the reduction in the concentration of that compound due to the combined action of H₂O₂ and irradiation. The height of each bar is related to the concentration of H₂O₂ introduced prior to irradiation. A greater concentration of H₂O₂ leads to a greater breakdown of the pharmaceutical compound.

Further tests have been performed at the National Ornamental Research Site-Dominican University California (NORS-DUC) which is a national facility for research on pathogens of ornamental plants. Under strictly controlled conditions, quantities of water were dosed with the quarantine pathogen Phytophthora ramorum. The water was then introduced into a treatment tube having a capacity of 5 gallons at rate of 50 gallons per minute and a dwell time of around 10 seconds. The water was irradiated with 12 UV tubes each of power output of 75 W. Due to the highly-contagious nature of this pathogen, the irradiated water was tested at the laboratories of the NORS-DUC test facility by the resident Staff Scientists. The results of these tests revealed near-total elimination of this pathogen from the irradiated water. Specifically, the concentration of this pathogen dropped from a concentration of 279,000 Colony-Forming Units per milliliter (CFU/ml) in the inlet water to a concentration of 9 CFU/ml in the irradiated water.

### 5. Advantages

One or more embodiments of the present system may provide one or more benefits over conventional UV treatment systems. These benefits may include:

1. Higher inactivation efficiency. The strong mixing of the liquid induced by the vortex, together with the presence of the air core, may ensure that all the inlet flow will be exposed to uniform UV radiation. Moreover, the increasing diameter of the air core may reduce the water depth in the rising column, particularly near the top of the tube. By careful selection of the tube height, tube diameter, bleed port diameter, bleed flow rate through the Venturi nozzle, and entry flow rate it may be possible to ensure that the water depth does not exceed the UV penetration depth.

2. Reduced energy consumption. Because of the reduction in water depth due to the formation of air core in the vortex, it may be possible to deliver the required UV dose using fewer UV lamps. These lamps may also be shorter than the conventional ones as they may need to cover only a limited region of the flow (see FIGS. 1-2). In addition, energy is saved as the UV lamps are not immersed in water and thus do not cause hydraulic losses.

3. Reduced maintenance. The forces generated by the vortex against the inner surface of the treatment tube reduce or eliminate build-up of materials and fouling of the inside of the treatment tube, thus reducing or eliminating the need to clean the tubes. Further, the UV tubes are easily accessed for replacement, and their electric connections are not in contact with the liquid.

4. Improved performance in the presence of suspended solids. Suspended solids that are present in the untreated water undergo the motions of swirl, rotation, and tumble as they travel upwards, thereby exposing pathogens that may have attached or embedded in them to UV radiation.

What is claimed is:

1. An apparatus for disinfecting a liquid with UV radiation, the apparatus comprising:
a treatment tube, wherein at least a portion of the treatment tube is transparent to UV light;
at least one inlet in the bottom portion of the treatment tube that is configured to direct the liquid into the treatment tube in a direction suitable for generating a vortex;
at least one outlet configured to allow disinfected liquid to exit the tube; and
at least one UV light source located external to the treatment tube, wherein the UV light source is configured so as not to contact the liquid,
and wherein the apparatus is configured to allow generation of a liquid vortex having an air core that extends towards the bottom of the treatment tube along the central axis of the treatment tube.

2. The apparatus of claim 1, wherein the apparatus is configured to enable the air core to extend all the way to the bottom of the treatment tube.

3. The apparatus of claim 1, wherein the treatment tube comprises:
a cylinder that is open at both ends; and
a base on which the cylinder may be placed, wherein the base comprises a bleed port to allow formation of the air core.

4. The apparatus of claim 1, wherein the treatment tube comprises a cylinder having a floor, and wherein the floor comprises a bleed port that is configured to allow formation of the air core.

5. The apparatus of claim 3, wherein the bleed port comprises a circular opening.

6. The apparatus of claim 5, wherein the circular opening comprises a rim that is raised above the surface of the base.

7. The apparatus of claim 1, wherein the flow rate of the liquid at the inlet may be adjusted to control the thickness of the liquid in the vortex between the interior surface of the tube and the exterior surface of the air core.

8. The apparatus of claim 1, further comprising a pump, wherein the pump supplies the liquid to the inlet.

9. The apparatus of claim 1, wherein the liquid is directed tangentially into the treatment tube.

10. The apparatus of claim 1, wherein the treatment tube is cylindrical.

11. The apparatus of claim 1, wherein the treatment tube comprises an outer column and an inner column, and wherein the outer column surface is not transparent to UV light, and wherein the outer column has one or more cutout sections to reveal the inner column, and wherein the inner column is transparent to UV, and wherein the inner column has the capability to rotate.

12. The apparatus of claim 1, wherein the UV light source is a straight rod.

13. The apparatus of claim 1, wherein the UV light source is toroidal.

14. The apparatus of claim 1, wherein the liquid is water.

15. The apparatus of claim 4, wherein the base is configured to allow injection of an oxidizing agent.

16. The apparatus of claim 1, wherein the inlet receives the liquid from a pump.

17. The apparatus of claim 1, wherein the inlet receives the liquid from an elevated reservoir.

18. A method for disinfecting a liquid with UV radiation, the method comprising:
injecting the liquid into an inlet at the bottom of a treatment tube, wherein the liquid is injected with a direction and flow rate that causes a vortex in the treatment tube, and wherein the vortex has an air core along the central axis of the treatment tube that extends towards the bottom of the treatment tube;
irradiating the liquid in the treatment tube with UV light, wherein the UV light source is located outside of the treatment tube; and
collecting the irradiated liquid from the top of the treatment tube.
